(19) United States
(12) Patent Application Publication
(10) Pub. No.: US 2019/0201418 A1
(43) Pub. Date: Jul. 4, 2019

(54) TREATMENT OF UTERINE FIBROIDS BY INTRAVAGINAL ADMINISTRATION OF A LOW DOSE OF SELECTIVE PROGESTERONE RECEPTOR MODULATOR (SPRM), ANTI-PROGESTIN, OR ANTI-PROGESTATIONAL AGENT

(71) Applicant: ARSTAT, INC., Flemington, NJ (US)

(72) Inventor: Arkady RUBIN, Flemington, NJ (US)

(73) Assignee: ARSTAT, INC., Flemington, NJ (US)

(21) Appl. No.: 16/295,577

(22) Filed: Mar. 7, 2019

Related U.S. Application Data
(63) Continuation of application No. 14/017,524, filed on Sep. 4, 2013, now abandoned.
Application No. 14/017,524 is a continuation of application No. PCT/US2011/063488, filed on Dec. 6, 2011.
(60) Provisional application No. 61/450,991, filed on Mar. 9, 2011.

(51) Int. Cl. A61K 31/575; A61K 31/567; A61K 9/00

(52) U.S. Cl. CPC A61K 31/575; A61K 31/567; A61K 9/0092; A61K 9/0039; A61K 9/0036

(57) ABSTRACT

A method and intravaginal drug delivery device for reducing size of a uterine fibroid in a female in need thereof are provided. The method includes administering intravaginally to the female a therapeutically effective amount of an active agent, wherein the agent is delivered on a delivery device directly to uterine fibroid, wherein said amount is able to significantly reduce the size of the uterine fibroid, wherein the active agent is any one of selective progesterone receptor modulator (SPRM), an anti-progestin agent, and an anti-progestational agent.

### CROSS REFERENCE TO RELATED APPLICATIONS

This application is a continuation of U.S. patent application Ser. No. 14/017,524 filed Sep. 4, 2013 which is a continuation of international application PCT/US2011/063488 filed Dec. 6, 2011, which claims the benefit of U.S. provisional patent application No. 61/450,991 filed Mar. 9, 2011, the contents of which are incorporated by reference.

### TECHNICAL FIELD

The present invention relates to the treatment of uterine fibroids and related symptoms. More specifically, the present invention relates to the pharmacological treatment of uterine fibroids by intravaginal administration of low doses of a selective progesterone receptor modulator (SPRM), anti-progestin, or anti-progestational agent.

### BACKGROUND

Uterine fibroids are common. They include fibromyomas, myomas, fibromas, leiomyomata, etc. and are classified into submucosal, intramural, and subserosal fibroids (see, for example, reference 6). The estimated prevalence of uterine fibroids in women of reproductive age ranges from 25% to 40%.^{1,2} An estimate from the National

Uterine Fibroids Foundation is much higher: as many as 80% of women in the United States have uterine fibroids; 25% of those women complain of fibroid-related symptoms severe enough to require treatment.^{3,4}

Uterine fibroids may be associated with a number of symptoms, including heavy menstrual bleeding (sometimes accompanied by anemia), menstrual pain, pelvic or abdominal pressure, pain during intercourse and obstructive symptoms, including increased frequency of urination (due to diminished bladder capacity) and constipation.^{5,6} Fibroids may also cause infertility. These symptoms are correlated with the size, number, location and occasional degeneration of the fibroids. Symptomatic uterine fibroids are most common in women aged from 35 to 50 years. It is reported that over 20 million women in the United States and Canada suffer from symptomatic uterine myomas.⁷

Women with severe symptoms often require appropriate therapy. Heavy menstrual bleeding (menorrhagia), may be treated with a non-hormonal medication, tranexamic acid, marketed in the U.S. as Lysteda® and both within and outside the U.S. as Cyklokapron®. Women with heavy menstrual bleeding are also frequently offered off-label use of approved hormonal contraceptives. For a number of women, the treatments may not be acceptable due to known contraindications, hormone-related adverse events and/or undesirable changes in the menstrual bleeding pattern, including unpredictable intra-cyclic bleeding, irregular menstrual periods and/or development of amenorrhea.^{8,9} For women having fibroids with distortion of the uterine cavity, insertion of the LNG-IUS (Mirena®) is not recommended. Women with painful menstrual periods may take analgesics for the temporary relief of this fibroid-induced symptom. None of the medications described above will cause fibroids to shrink, and thus none can be viewed as a permanent treatment option.

Surgical removal of fibroids seems like a definitive solution to the problem. Surgical options include myomectomy (abdominal, laparoscopic or hysteroscopic) and hysterectomy. However, myomectomies may result in postoperative wound infection, injuries to internal organs, internal scarring and bleeding. The uterus may be weakened after surgery, and cesarean section may be required for the delivery of future pregnancies. Importantly, myomectomies do not prevent fibroid re-growth, particularly in young women.¹⁰ Recurrent symptoms and subsequent procedures cannot be ruled out. Hysterectomy is a major surgical procedure used to permanently resolve the fibroid-related symptoms. However, removal of the uterus is a radical treatment option with known undesirable characteristics, including loss of fertility, surgical morbidity and high cost.

Another surgical procedure, uterine artery embolization (UAE, also called uterine fibroid embolization or UFA) does not remove fibroids. It blocks the blood flow to fibroids causing them to shrink in size. Uterine artery embolization leads to the significant reduction in the overall volume of the fibroids as well as that of the uterus (approximately 40%-50% and 30%-40%, respectively). It is reported that 80%-90% of patients have improvement in symptoms. However, for some patients this treatment is ineffective. Possible serious complications—injury to a vessel, uterine infection, blood clots and injury to the ovaries—need to be considered as well.¹¹

Gonadotropin-releasing hormone agonists (GnRHa) may also be used to reduce the size of the uterine fibroids and alleviate related symptoms. This group of drugs includes goserelin (Zoladex®), buserelin, a monthly injection of leuprolide (depot Lupron®) and nafarelin (Synarel®) nasal spray. The drugs are effective and can reduce fibroids' size by 30-90%. However, they are associated with a number of significant and distressing adverse events, including menopause-like symptoms (e.g., hot flashes, night sweats, vaginal dryness, loss of bone density, weight gain and depression). A recommended use for these drugs is for a short period of time in women nearing menopause and/or prior to planned uterine surgery.⁶

The lack of safe and effective medications leads to the high prevalence of the surgical procedures described earlier. The development of non-invasive treatments targeting both the size of fibroids and their related symptoms has consequently been gaining significant clinical and public health importance.

The current research activities focus on a class of drugs called selective progesterone receptor modulators (SPRMs). These drugs act on progesterone receptors and display progesterone antagonist or mixed agonist/antagonist activity.¹² The oldest member of this class, mifepristone, is considered as a progesterone antagonist (anti-progestin, anti-progestational agent). Numerous compounds in this pharmacological class have been synthesized; some of them have been tested in clinical trials. Phase III studies have confirmed the efficacy and safety of oral (5-mg and 10-mg tablets) ulipristal acetate (Esmyal, a SPRM, in the treatment of symptomatic uterine fibroids.¹³

The mechanism of action of SPRMs and anti-progestins is not completely understood. Studies have suggested that leiomyomata growth is steroid-dependent and that mitotic activity in leiomyomata is greater in the luteal phase of the menstrual cycle. There is growing biochemical, histologic and clinical evidence that progesterone has a critical role in leiomyoma growth. As a progesterone antagonist, mifepristone decreases the number of progesterone receptors in the myometrium and in leiomyomata. Estrogen-dependent endometrial proliferation, mitotic and secretory activities are suppressed, and endometrial thickness and wet weight are reduced.¹² Suppression of ovarian activity and maintenance of a hormonal milieu similar to that of the early follicular phase may be another factor. Finally, mifepristone may cause alterations in blood flow to the leiomyomata by a direct vascular effect.¹⁴ Other pharmacodynamic properties of SPRMs and anti-progestins (such as direct effects on the endometrial vasculature and inhibition of ovulation) may also contribute to the development of amenorrhea^{12,14}, considered beneficial for women with uterine fibroids.

The pharmacology, mechanisms of action and possible clinical applications of mifepristone are described in numerous publications (see, for example, reference number 15).

A number of clinical studies have evaluated the efficacy and safety of oral mifepristone in the treatment of uterine fibroids and related symptoms.^{14,17,18,19,20,21} The data suggests that 10 mg/day or higher oral doses of mifepristone are effective in the reduction of uterine and fibroid volumes and related symptoms. However, at this dose, the incidence of side effects such as endometrial hyperplasia, hot flashes and elevated liver enzymes seems to be greater than desired. Two clinical programs of other SPRMs were discontinued for safety reasons. Phase III studies of oral asoprisnil were terminated due to changes in the endometrial lining of the uterus that resulted in additional invasive procedures in some patients. Phase Ill studies of another compound of the same class—oral CDB-4124 (Proellex®)—were suspended because of significant increases in liver enzymes.

While a 5 mg oral mifepristone dose may be a safer alternative, its ability to reduce uterine volume and eliminate some of the fibroid-related symptoms is limited. A further oral dose reduction (2.5 mg/day) may be harder to justify due to an appreciably smaller reduction of uterine volume and a lower incidence of amenorrhea, with no noted benefits of therapy extending beyond the initial three months.²¹ Taken together, the clinical evidence indicates that the efficacy of oral mifepristone (or another SPRM, anti-progestin, or anti-progestational agent) in the treatment of uterine fibroids must be weighed against the potentially disturbing side effects associated with this medication.

Effectiveness and safety of intravaginal drug delivery is supported by a substantial body of evidence. It is established for estrogens used to treat vaginal atrophy and related symptoms²³, as well as osteoporosis and other menopausal symptoms.²⁴ Other examples of compounds efficacious after vaginal administration include (but are not limited to) misoprostol for cervical ripening²⁵, a danazol ring for the treatment of infiltrating endometriosis²⁶, and a progesterone gel.^{27,28} The contraceptive efficacy of levonorgestrel(LNG)-containing intrauterine system, Mirena® with 20 mcg/day LNG delivery is at least comparable to that reported for the LNG-only pill delivering a 50% greater daily dose. As noted in the Mirena NDA Medical review, serum concentrations of levonorgestrel for Mirena are approximately one-tenth the serum concentration produced by an oral contraceptive containing 0.1 mg LNG and about half that produced by the Norplant® system. The local endometrial concentrations, however, are over 100 times higher in Mirena users than in users of oral contraceptives containing 0.25 mg LNG.²⁹

### SUMMARY

The present invention provides a method for effectively reducing the size of uterine fibroids or the uterus and/or improving fibroid-related symptoms without the undesirable side effects of oral medications by providing for intravaginal delivery of a selective progesterone receptor modulator (SPRM), anti-progestin, or anti-progestational agent at doses which are significantly lower than oral doses known in the art. As disclosed herein, local administration of SPRM, or anti-progestin, or anti-progestational agent can be rendered safe and efficacious if it utilizes vaginal drug delivery. Drug delivery devices useful in the method of the present invention allow for drug delivery to the affected tissues (e.g., vagina and adjacent organs, including uterine fibroids). While a vaginal ring is a preferred drug delivery device in the method of the present invention, other delivery devices can be also used. Specifically, an intrauterine device (IUD) designed for insertion in women with fibroids could be considered.

According to the present invention, the active drug (i.e., SPRM, anti-progestin, or anti-progestational agent) is delivered directly to the affected tissue(s), particularly the uterine fibroids that are close to the vagina where the delivery device (e.g., vaginal ring or IUD) is placed. As specified herein, effective local concentrations of drug are achievable with doses much lower than those administered by the oral route. When used according to the method of the invention, levels of the SPRM (or anti-progestin, or anti-progestational agent) in systemic circulation are greatly reduced as compared to oral therapies, possibly below detectable limits, leading to a lower incidence of adverse events.

The intravaginal administration of mifepristone (or another SPRM, anti-progestin, or anti-progestational agent) according to the method of the present invention substantially reduces, and possibly eliminates, first-pass hepatic metabolism. This may alleviate abnormalities in the liver function tests noted earlier. Since mifepristone has anti-glucocorticoid properties (with glucocorticoid blockade reported at doses ≥50 mg²²), reduced systemic circulation of the drug ensures better control of plasma cortisol levels. Relatively high local tissue concentrations of mifepristone (or another SPRM, anti-progestin, or anti-progestational agent) achievable by the method of the present invention ensure a faster reduction in size of the fibroids or the uterus, as well as a faster improvement in related symptoms. Studies of oral mifepristone tablets suggest a treatment period ranging from 3 to 6 months.^{14,17,18,19,20,21} Intravaginal delivery targeting local tissues enables a shorter duration of therapy. A shorter treatment course is expected to reduce the incidence of hyperplastic endometrial changes attributed to prolonged exposure to some anti-progestins. The intravaginal administration of mifepristone (or another SPRM, anti-progestin, or anti-progestational agent) may also prevent fibroid(s) re-growth. Better compliance (thereby avoiding missed pills by women using such a device) is also expected.

For a number of drugs delivered intravaginally, the oral route/intravaginal route ratio for the daily doses seems to be close to 10:1. The same ratio may reasonably be assumed for mifepristone (or another SPRM, anti-progestin, or anti-progestational agent).

While the exact intravaginal doses for each drug useful in the method of the present invention are going to be determined in clinical trials, the possibility of a drastic dose decrease, relative to the currently tested oral doses, with no compromise (but rather improvement) in the reduction in size of the fibroids or the uterus and in relief of related symptoms, is surprising and new. With an expected decrease in drug-related adverse events, this treatment modality may be considered as the first treatment option in the management of uterine fibroids.

Also surprising and new is the possibility of achieving a therapeutic effect (manifested in a reduction in size of both the fibroids and the uterus and in relief of related symptoms) in the absence of detectable plasma concentration levels, or in the presence of circulating levels of the drug much lower than those reported after oral administration of the same compounds.

In addition to mifepristone, the reduction in size of the fibroids or the uterus and relief of related symptoms may utilize a number of agents from a class of drugs called selective progesterone receptor modulators (SPRM), or from the class of drugs called anti-progestins, or from the class of drugs called anti-progestational agents, including, but not limited to mifepristone, ulipristal acetate, asoprisnil, onapristone, CDB-2914, CDB-4124 and metabolites thereof. The exact doses of these compounds will be determined in clinical trials. Initial dose selection will be driven by a number of factors, including, but not limited to, the potency of the compound tested, the number and size of the uterine fibroids and the severity of the symptoms associated with uterine fibroids), as well as patient characteristics (age, weight, duration of the disease, etc).

Specific dose regimens (e.g., continuous without interruptions, or drug-administration period(s) followed by intermittent drug-free interval(s) when the drug delivery device is removed) are also going to be determined in clinical trials.

### DETAILED DESCRIPTION

The embodiments disclosed herein are only examples of the many possible advantageous uses and implementations of the innovative teachings presented herein.

In general, statements made in the specification of the present application do not necessarily limit any of the various claimed embodiments. Moreover, some statements may apply to some inventive features but not to others.

### Definitions:

A vaginal ring (also known as an intravaginal ring) is a polymeric drug delivery device providing controlled release of drug(s) to the vagina and adjacent organs, including uterine fibroids over an extended period of time.

An Intrauterine Device (also known as an IUD) is an object placed in the uterus to prevent pregnancy. In the method of the present invention, the medicated IUD is considered as a drug delivery device providing controlled release of drugs to the vagina and adjacent organs, including uterine fibroids over an extended period of time.

A therapeutically effective amount of SPRM, or anti-progestin, or anti-progestational agent is defined as the amount of a drug that results in a significant (preferably, at least 15%) reduction in size of uterine fibroids when compared to the pre-treatment levels.

This invention provides for intravaginal delivery of a therapeutically effective amount of Selective Progesterone Receptor Modulator (SPRM), anti-progestin, or anti-progestational agent for the reduction in size of the uterine fibroids or the uterus and improvement in related symptoms.

In the method of the present invention, the SPRM, anti-progestin, or anti-progestational agent can be delivered using any intravaginal delivery device known in the art. Non-limiting examples of useful delivery devices include vaginal ring, intrauterine device, and vaginal tablet.

In a preferred embodiment, the drug delivery device is a vaginal ring. In another preferred embodiment, the drug delivery device is a medicated intrauterine device (IUD). In one embodiment, the agent can be mixed throughout the vaginal ring. In another embodiment, the agent can be distributed uniformly throughout the vaginal ring. In yet another embodiment, the agent can be encapsulated in a part of the vaginal ring. In a further embodiment, the agent can be located at the center of the vaginal ring. In yet another embodiment, a membrane of the agent can be placed between an un-medicated core and a metering layer of appropriate material.

The use of a vaginal drug delivery device delivering the agent directly to the affected tissues (e.g., vagina and adjacent organs, including uterine fibroids) is expected to enhance the agent's efficacy in the reduction in size of the fibroids and the uterus and improvement in related symptoms; it may also result in a shorter duration of therapy compared to other routes of drug administration.

The use of a vaginal drug delivery device delivering the agent directly to the affected tissues is also expected to significantly reduce the agent's daily dose when compared to other routes of drug administration; this may result in a lower systemic drug circulation, possibly below detectable levels, and a lower incidence of drug-related adverse events.

In all embodiments, the agent is from a class of drugs called selective progesterone receptor modulators (SPRM), or from the class of drugs called anti-progestins, or from the class of drugs called anti-progestational agents. Non-limiting examples of useful agents include, e.g., mifepristone, ulipristal acetate, asoprisnil, onapristone, CDB-2914, CDB-4124, and metabolites thereof.

In one embodiment, daily agent doses useful in the method of the present invention do not exceed 1.4 mg. In a preferred embodiment, daily agent doses useful in the method of the present invention range from 50 mcg to 1 mg. In another preferred embodiment, the agent is mifepristone with a daily drug delivery dose ranging from 100 mcg to 500 mcg. In yet another preferred embodiment, the agent is CDB-4124 with a daily drug delivery dose ranging from 150 mcg to 600 mcg. In a further preferred embodiment, the agent is ulipristal acetate with a daily drug delivery dose ranging from 200 mcg to 700 mcg.

In certain embodiments, the method of the invention is used to treat females with symptomatic uterine fibroids. Non-limiting examples of symptoms include, e.g., heavy menstrual bleeding, menstrual pain, pelvic and abdominal pressure, pain during intercourse and obstructive symptoms, such as urinary flow frequency and constipation. In certain embodiments, the method of the invention is used to treat females with non-symptomatic uterine fibroids. In certain other embodiments, the method of the invention is used to treat females with uterine fibroids clinically diagnosed with menorrhagia. In certain additional embodiments, the method of the invention is used to treat females with uterine fibroids clinically diagnosed with anemia. In certain further embodiments, the method of the invention is used to treat females with uterine fibroids clinically diagnosed with dysmenorrhea. In certain other embodiments, the method of the invention is used to treat females without interruption of drug delivery with a treatment period ranging from two weeks to six months. In certain additional embodiments, the method of the invention is used to treat females without interruption of drug delivery with a treatment period ranging from six months to three years. In a preferred embodiment, the method of the invention is used to treat females without interruption of therapy with a treatment period ranging from approximately one month to approximately three months. In certain other embodiments, the method of the invention is used to treat females with the periods of drug delivery (ranging from approximately one month to approximately three months) followed by the drug-free intervals when the drug delivery device is removed. In certain further embodiments, upon administration of the agent according to the method of the invention, the amount of the agent in the female's systemic circulation is below detection levels.

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

Example 1: The vaginal ring serving as a drug delivery device comprises a supporting ring free of active drug. The next (second) layer contains medication selected for treatment of uterine fibroids (selective progesterone receptor modulator, or anti-progestin, or anti-progestational agent). This layer is coated with the third, drug-free layer. Detailed description of such vaginal ring and suitable manufacturing methods can be found in U.S. Pat. No 4,822,616.

Per U.S. Pat. No 4,822,616, the supporting ring is made from a physiologically acceptable synthetic resin, such as, e.g., polyethylene, RTV silicone elastomers, LTV silicone elastomers, polyamides and polytetrafluoroethylene. The second layer with active medication comprises a pharmaceutically acceptable resin from which the drug is released. A preferred embodiment consists of the combination of drug and LTV silicone elastomer with a composition also described in the patent. Any LTV silicone elastomer is used in the third layer. The proposed vaginal ring ensures release of the active drug within the limits of the dosage required for the desired reduction in size of both uterine fibroids and the uterus.

In one embodiment, the second layer is medicated with mifepristone in an amount adequate to release the drug in a rate of 250-300 mcg/day. In another embodiment, the second layer is medicated with CDB-4124 in an amount adequate to release the drug in a rate of 300-400 mcg/day. In yet another embodiment, the second layer is medicated with ulipristal acetate in an amount adequate to release the drug in a rate of 300-500 mcg/day.

In all described embodiments, the treatment is continuous without interruption. A preferred duration of therapy (following insertion of the ring) ranges from approximately one month to approximately three months.

Example 2: The vaginal ring serving as a drug delivery device comprises the active drug selected for treatment of excessive menstrual blood loss (tranexamic acid or another antifibrinolytic or hemostatic agent)and a delivery module. Delivery module comprises (a) reservoir for storing the active drug, (b) a rate controller or wall that is formed of styrene-butadiene copolymer that maintains the prescribed rate of drug release throughout the life of system, (c) energy source or the concentration of active drug in reservoir that provides the driving means for transferring the active drug from a higher amount in reservoir to the rate controller, (d) an inner mass transfer conductor for housing the active drug in reservoir, and (e) a portal that provides the exit from the drug delivery module to the tissues. Detailed description of such vaginal ring and its manufacturing process can be found, for example, in U.S. Pat. No 4,250,611.

In one embodiment, the delivery module of the vaginal ring contains mifepristone in an amount supporting the drug release at a rate of 250-300 mcg/day. In another embodiment, the delivery module of the vaginal ring contains CDB-4124 in an amount supporting the drug release at a rate of 300-400 mcg/day. In yet another embodiment, the delivery module of the vaginal ring contains ulipristal acetate in an amount supporting the drug release at a rate of 300-500 mcg/day.

In all described embodiments, the treatment is continuous without interruption. A preferred duration of therapy (following insertion of the ring) ranges from approximately one month to approximately four months.

Example 3: The vaginal ring serving as a drug delivery device is a ring-shaped solid carrier made of silicone rubber (polysiloxane) or other suitable material. The ring has a homogenous design with an active drug dispersed in the carrier. Detailed description of such vaginal ring can be found, for example, in U.S. Pat. No 5,869,081. Per U.S. Pat. No 5,869,081, the vaginal ring provides sustained release of the medication and results in low circulatory levels of the drug, while concentrating its biological effect on a regional level.

In one embodiment, the carrier contains mifepristone in an amount supporting the drug release at a rate of 250-300 mcg/day. In another embodiment, the carrier contains CDB-4124 in an amount supporting the drug release at a rate of 300-400 mcg/day. In yet another embodiment, the carrier contains ulipristal acetate in an amount supporting the drug release at a rate of 300-500 mcg/day.

In all described embodiments, the treatment is continuous without interruption. A preferred duration of therapy (following insertion of the ring) ranges from approximately two weeks to approximately one month.

Example 4: The medicated intrauterine device (IUD) serving as a drug delivery device is inserted into the uterus for a predetermined time period. The device comprises a body of the device in combination with an external surface contacting the uterus. The external surface is medicated and provides controlled drug release. Detailed description of such IUD can be found in U.S. Pat. No. 4,359,046.

In one embodiment, the IUD is medicated with mifepristone in an amount supporting the drug's release at a rate of 200-250 mcg/day. In another embodiment, the carrier contains CDB-4124 in an amount supporting the drug release at a rate of 250-300 mcg/day. In yet another embodiment, the carrier contains ulipristal acetate in an amount supporting the drug release at a rate of 250-300 mcg/day.

Contraceptive action of the IUD is considered as optional. In all described embodiments, the treatment is continuous without interruption. A preferred duration of therapy (following insertion of the IUD) is up to three years.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

¹ Shwalisz K., et al. Effectiveness of Asoprisnil, a Selective Progesterone Receptor Modulator (SPRM) in Treating Uterine Leiomyomata. Advances in Leiomyoma Research, 2^{nd} NIH International Congress, Feb. 24-25, 2005, Bethesda, Md.

² Kaunitz A M. Progestin-releasing intrauterine systems and leiomyoma. Contraception. 2007 June;75(6 Suppl):S130-3. Epub 2007 Mar. 9.

³ About Uterine Fibroids. Introduction. National Uterine Fibroids Foundation, accessed at http://www.nuff.org/health.htm (reference on file)

⁴Viswanathan M. et al. Management of uterine fibroids: an update of the evidence, Evid Rep Technol Assess (Full Rep). 2007 July;(154):1-122.

⁵ Rodriguez M I, Darney P D. Non-contraceptive applications of the levonorgestrel intrauterine system. International Journal of Women's Health. Volume 2010:2 P. 63-68.

⁶ ADAM Mobile Encyclopedia Uterine Fibroids. Accessed at http://pfizer.adam.com/content.aspx?productId=101&pid=1&gid=000914 (reference on file)

7 Preglem licenses additional rights for ulipristal acetate from HRA Pharma. Accessed at http://www.preglem.com/files newspdf/PR_Preglem%20NA%20Rights%20to%20Uliprist al%20Acetate.pdf (reference on file)

⁸ David Chelmow Non-surgical Options for Menorrhagia. OBG Management November 2005 (Vol. 17, No.11)

⁹ National Collaborating Centre for Women's and Children's Health. Heavy menstrual bleeding. London (UK): Royal College of Obstetricians and Gynaecologists (RCOG); 2007 January.

¹⁰Rossetti A, Sizzi O, Soranna L, et al. Long-term results of laparoscopic myomectomy: recurrence rate in comparison with abdominal myomectomy. Hum Reprod 2001;16:770-774.

¹¹Uterine Artery Embolization. Wikipedia. Assessed at http://en.wikipedia.org/wiki/Uterine_artery_embolization (reference on file)

¹² Spitz I M., Mifepristone: where do we come from and where are we going? Clinical development over a quarter of a century. Contraception. Corrected Proof. 1 Feb. 2010.

¹³ Preglem announces positive Phase III results for Esmya™ as an effective treatment for uterine fibroids. Accessed at http://www.preglem.com/files/newspdf/PR_PregLem%20PEARLII_Phase%20III%20results_%20FINAL%20.pdf (reference on file)

¹⁴Steinauer, J, Pritts, E A, Jackson, R, Jacoby A F. Systematic review of mifepristone for the treatment of uterine leiomyomata. Obstet Gynecol 2004; 103:1331.

¹⁵Spitz I M. Pharmacology and mechanisms of action of progesterone receptor antagonists and selective progesterone receptor modulators. UpToDate. Online 18. 2, May 2010.

¹⁶Mifepristone. Wikipedia. Accessed at http://en.wikipedia.org/wiki/Mifepristone (reference on file)

¹⁷Eisinger S H., et al. Twelve-month safety and efficacy of low-dose mifepristone for uterine myomas. J Minim Invasive Gynecol 2005; 12:227.

¹⁸Gommier B, Magning G. Mifepristone for treatment of myomas [meeting abstract]. Third Symposium. Misoprostol and mifepristone in obstetrics and gynecology. Valencia, Spain, Oct. 4-5, 2007).

¹⁹Fiscella K, et al. Effect of mifepristone for symptomatic leiomyomata on quality of life and uterine size: a randomized controlled trial. Obstet Gynecol 2006;108:1381-7.

²⁰Esteve C J L, et al. Mifepristone for the treatment of uterine leiomyomas: a randomized controlled trial. Obstet Gynecol 2008;112:1029-36.

²¹Eisinger, S H, et al. Open-label study of ultra low-dose mifepristone for the treatment of uterine leiomyomata. Eur J Obstet Gynecol Reprod Biol 2009, 0ct. 1;146(2):215-8;

²²Fiscella K. Use of antiprogestins for treatment of uterine fibroids: clinical trials (presentation). Advances in Uterine Leiomyoma Research. 2^{nd} NIH International Congress. Feb. 24-25, 2005. Bethesda Md. (reference on file)

²³Kingsberg S A., et al. Treating dyspareunia caused by vaginal atrophy: a review of treatment options using vaginal estrogen therapy. International Journal of Women's Health. August 2009 , Volume 2009:1 Pages 105-111

²⁴Ballagh S A. Vaginal Ring Hormone Delivery Systems in Contraception and Menopause. CLINICAL OBSTETRICS AND GYNECOLOGY/VOLUME 44/NUMBER 1/MARCH 2001

²⁵Iyer V., et al. Vaginal drug delivery. ExpressPharma, 1-15 Jul. 2008

²⁶Igarashi M. Novel vaginal danazol ring therapy for pelvic endometriosis, in particular deeply infiltrating endometriosis. Hum Reprod. 1998 July;13(7):1952-6.

²⁷Ficicioglu C. et al. High local endometrial effect of vaginal progesterone gel. Gynecological Endocrinology, Volume 18, Issue 5 May 2004, pages 240-243.

²⁸Kuhl H. Pharmacology of estrogens and progestogens: influence of different routes of administration. Climacteric 2005; 8 (Suppl 1)3-63.

²⁹ Center for Drug Evaluation and Research. Application Number 21-225. Mirena® NDA

Medical review; accessed at Mirena® NDA Medical review. Accessed at http://www.accessdata.fda.gov/drugsatfda_docs/nda/2000/21-225.pdf_Mirena_Medr.pdf (reference on file)

What is claimed is:

1. A method for reducing size of a uterine fibroid in a female in need thereof comprising:
administering intravaginally to the female a therapeutically effective amount of an active agent, wherein the agent is delivered on a delivery device directly to uterine fibroid, wherein a daily dose of the active agent does not exceed 500 mcg, wherein the active agent is mifepristone.

2. The method of claim 1, wherein the delivery device is any one of: a vaginal ring, a medicated intrauterine device (IUD), and a vaginal tablet.

3. The method of claim 2, wherein the active agent contained in the vaginal ring is mixed throughout the vaginal ring.

4. The method of claim 3, wherein the active agent is delivered at a daily dose ranging from 100 mcg to 400 mcg.

5. The method of claim 1, wherein the female has non-symptomatic or symptomatic uterine fibroids, wherein uterine fibroids symptom comprises at least one of: heavy menstrual bleeding, menstrual pain, pelvic pressure, abdominal pressure, pain during intercourse, urinary flow frequency, constipation, menorrhagia, anemia, and dysmenorrhea.

6. The method according to claim 1, wherein the active agent is administered:
without interruption of drug delivery with duration of treatment period ranging from two weeks to six months, without interruption of drug delivery with duration of treatment period ranging from six months to three years, or in at least two periods of drug delivery ranging from approximately one month to approximately three months, separated by drug-free intervals when a drug delivery device is removed.

7. The method according to claim 1, wherein the active agent comprises any one of:
mifepristone and metabolites thereof.

8. The method of claim 7, wherein the daily dose of each of the active agents ranging from 100 mcg to 500 mcg.
